SEQUENCE LISTING (12 sequences)

SEQ ID NO: 1 - DNA, 42 bp, Artificial Sequence
  chemically-synthesized hybridization probe
taggactaccaggggttttcgcctgcaagtcctaagacgcca

SEQ ID NO: 2 - DNA, 47 bp, Artificial Sequence
  chemically-synthesized hybridization probe
cttcgttcgattgtctggacttataatgctgaacttctggtcaatcg

SEQ ID NO: 3 - DNA, 41 bp, Artificial Sequence
  chemically-synthesized hybridization probe
cttcgttcgattgtctggacttataatgctgaacttctggt

SEQ ID NO: 4 - DNA, 50 bp, Artificial Sequence
  chemically-synthesized hybridization probe
cttcgttcgattgtctggacttataatgctgaacttctggtcaatcgaac

SEQ ID NO: 5 - DNA, 45 bp, Artificial Sequence
  chemically-synthesized hybridization probe
gttacttcgttcgattgtctggacttataatgctgaacttctggt

SEQ ID NO: 6 - DNA, 41 bp, Artificial Sequence; identical to SEQ ID NO: 3
  chemically-synthesized hybridization probe

SEQ ID NO: 7 - DNA, 39 bp, Artificial Sequence
  chemically-synthesized hybridization probe
tcgttcgattgtctggacttataatgctgaacttctggt

SEQ ID NO: 8 - DNA, 59 bp, Artificial Sequence
  chemically-synthesized hybridization probe
catgctactcaacacagttaccatattccaattcacttttcataattgctggttgagta

SEQ ID NO: 9 - DNA, 15 bp, Artificial Sequence
  chemically-synthesized capture probe
gtgttgagtagcatg

SEQ ID NO: 10 - DNA, 52 bp, Artificial Sequence
  chemically-synthesized hybridization probe
acacgtgtaccttactgcagacaaagaatccactcaaaaggcaatggtacac

SEQ ID NO: 11 - DNA, 15 bp, Artificial Sequence
  chemically-synthesized capture probe
gtaaggtacacgtgt

SEQ ID NO: 12 - DNA, 18 bp, Artificial Sequence
  chemically-synthesized hybridization probe
ttnntagatccttaagga

(19) United States
(12) Patent Application Publication
(10) Pub. No.: US 2008/0193940 A1
(43) Pub. Date: Aug. 14, 2008

(54) SYSTEMS AND METHODS FOR DETECTING NUCLEIC ACIDS

(75) Inventors: Vissarion Aivazachvili, Oakland, CA (US); Kristian Scaboo, Castro Valley, CA (US); Eugene Spier, Palo Alto, CA (US)

Correspondence Address: MORRIS MANNING MARTIN LLP, 3343 PEACHTREE ROAD, NE, 1600 ATLANTA FINANCIAL CENTER, ATLANTA, GA, 30326, US

(73) Assignee: APPLERA CORPORATION, APPLIED BIOSYSTEMS GROUP, Foster City, CA (US)

(21) Appl. No.: 11/965,807

(22) Filed: Dec. 28, 2007

Related U.S. Application Data
(60) Provisional application No. 60/877,611, filed on Dec. 29, 2006.

(51) Int. Cl. C12Q 1/68

(52) U.S. Cl. 435/6

(57) ABSTRACT

A method and kit for detecting a target nucleic acid in a sample is described. The sample to be analyzed may include a primer which hybridizes to at least a portion of the target nucleic acid, a probe having a first region which hybridizes to at least a portion of the target nucleic acid and a second region having a detectable label, a polymerase which extends the hybridized primer and an enzyme comprising exonuclease activity that can cleave the hybridized hybridization probe to thereby generate a labeled probe fragment. At least one portion of the hybridization probe hybridizes to another portion of the hybridization probe to thereby form a folded structure. The method can involve melting the sample, reducing the temperature of the sample to allow primer and probe to each hybridize to at least a portion of single stranded target nucleic acid in the sample, elongating the primer and releasing the labeled probe fragment. The sample can be contacted with a solid support comprising surface bound capture probes which hybridize to the labeled probe fragments. The label can then be detected.

This application claims the benefit of Provisional U.S. Patent Application No. 60/877,611, filed on Dec. 29, 2006, which is incorporated by reference herein in its entirety.

Pursuant to the provisions of 37 C.F.R. § 1.52(e)(5), the sequence listing text file named 0037USU1_ST25, created on Dec. 27, 2007 and having a size of 3,027 bytes, and which is being submitted herewith, is incorporated by reference herein in its entirety.

The section headings used herein are for organizational purposes only and should not be construed as limiting the subject matter described herein in any way.

### FIELD

This application relates generally to methods and systems for detecting biological molecules and, in particular, to methods and systems for detecting nucleic acids in a sample.

### INTRODUCTION

Nucleic acid amplification may be performed in conjunction with a variety of assays. Such assays may be qualitative, for example when used to evaluate a biological sample. However, a wide variety of biological applications could be improved by the ability to detect the amplification of target nucleic acids, without requiring either cumbersome blotting techniques, or the expensive and delicate equipment typically required for optical methods.

Accordingly, there still exists a need for improved methods for detecting nucleic acids in a sample.

### SUMMARY

According to a first embodiment, a method for detecting a target nucleic in a sample is provided which comprises:

melting the sample by heating the sample to a first temperature, wherein the sample comprises:

a primer which hybridizes to at least a portion of the target nucleic acid;
a hybridization probe comprising first and second regions, wherein the first region hybridizes to at least a portion of the target nucleic acid and the second region does not hybridize to the target nucleic acid and wherein the second region comprises a detectable label; and
a polymerase and an enzyme comprising an exonuclease activity wherein the polymerase extends the hybridized primer in the direction of the hybridized probe and the exonuclease activity of the enzyme cleaves the hybridized probe to thereby release a probe fragment comprising the second region of the probe and the detectable label; and
   wherein the first temperature is above the Tₘ of the primer and double stranded nucleic acid present in the sample;

subsequently annealing the sample by reducing the temperature to a second temperature lower than the first temperature to allow the primer and the hybridization probe to each hybridize to a single stranded portion of the target nucleic acid in the sample; and

subsequently elongating the primer by allowing the polymerase to extend the primer hybridized to the target nucleic acid at a third temperature;

allowing the exonuclease activity of the enzyme to cleave the hybridization probe thereby releasing the probe fragment;

optionally repeating melting, annealing and elongating at least once;

contacting the sample with a surface of a solid support, wherein the surface of the solid support comprises one or more capture probes which hybridize to at least a portion of the second region of the probe fragment;

allowing the capture probes to hybridize to at least a portion of the probe fragment present in the sample at a fourth temperature wherein the fourth temperature is lower than the second and third temperatures; and

detecting label on the surface of the solid support;

wherein at least one portion of the hybridization probe hybridizes to another portion of the hybridization probe to thereby form a folded structure and wherein the melting temperature (Tₘ) of the folded structure is lower than the third temperature and higher than the fourth temperature.

According to a second embodiment, a kit for detecting a target nucleic acid in a sample is provided which comprises:

a hybridization probe comprising a first region which hybridizes to at least a portion of the target nucleic acid and a second region comprising a detectable label, wherein the second region does not hybridize to the target nucleic acid and wherein an exonuclease enzyme can cleave the hybridization probe when hybridized to the target nucleic acid to thereby produce a probe fragment comprising the second region and the detectable label;

a solid support comprising a capture probe on a surface thereof, wherein the capture probe hybridizes to the second region of the probe fragment;

optionally, a primer which hybridizes to at least a portion of the target nucleic acid; and

optionally, a polymerase and an enzyme comprising an exonuclease activity wherein the polymerase extends the hybridized primer in the direction of the hybridized probe and the exonuclease activity of the enzyme cleaves the hybridized probe to thereby release a probe fragment comprising the second region of the probe and the detectable label;

wherein at least one portion of the hybridization probe hybridizes to another portion of the hybridization probe to thereby form a folded structure and wherein the melting temperature (Tₘ) of the folded structure is lower than the melting temperature of the duplex formed when the intact hybridization probe hybridizes to the target nucleic acid and higher than the melting temperature of the duplex formed when the probe fragment hybridizes to the capture probe.

### BRIEF DESCRIPTION OF THE DRAWINGS

The skilled artisan will understand that the drawings, described below, are for illustration purposes only. The drawings are not intended to limit the scope of the present teachings in any way.

FIG. 1A is a schematic for the design of the components and the steps of an assay that uses a hybridization probe capable of forming a folded structure, wherein the hybridization probe can be hybridized to a target sequence, wherein a portion of the hybridized probe is cleaved to form a labeled probe fragment and wherein the labeled probe fragment can be captured and detected on a surface (e.g. using an electrode surface).

FIG. 1B is an illustration showing the predicted folded structure of a hybridization probe which has a predicted Tₘ of 61.7° C.

FIG. 2 is a bar chart showing the electrochemical signal generated by the hybridization probe having the nucleotide sequence illustrated in FIG. 1B after 40 cycles of polymerase chain reaction (PCR) at various times and temperatures.

FIG. 3A is an illustration showing the predicted folded structure of a hybridization probe which has a predicted Tₘ of 43.9° C.

FIG. 3B is a bar chart showing the electrochemical signal generated by the hybridization probe having the nucleotide sequence illustrated in FIG. 3A.

FIG. 4 is an illustration showing the predicted folded structure of a hybridization probe which has a predicted Tₘ of 34.2° C. wherein the hybridization probe differs from the probe illustrated in FIG. 3A in that 6 3′-nucleotides are removed from the probe shown in FIG. 3A.

FIG. 5A is a bar chart showing electrochemical signal generated by the hybridization probe having the nucleotide sequence illustrated in FIG. 3A.

FIG. 5B is a bar chart showing electrochemical signal generated by the hybridization probe having the nucleotide sequence illustrated in FIG. 4.

FIG. 6A is an illustration showing the predicted folded structure of a hybridization probe which has a predicted Tₘ of 53.1° C. wherein the probe has a predicted 3′ 9 base double stranded region in contrast to the predicted 3′ 6 base double stranded region of the probe illustrated in FIG. 3A.

FIG. 6B is a bar chart showing electrochemical signal generated by the hybridization probe having the nucleotide sequence illustrated in FIG. 3A.

FIG. 6C is a bar chart showing electrochemical signal generated by the hybridization probe having the nucleotide sequence illustrated in FIG. 6A.

FIG. 7A is a bar chart showing electrochemical signal generated by a hybridization probe having the nucleotide sequence:

(SEQ ID NO. 5)

GTTACTTCGTTCGATTGTC^{▾}TGGACTTATAATGCTGAACTTCTGGT

wherein the probe has a 19 mer nucleotide sequence that is non-complementary to a target sequence of a target nucleic acid.

FIG. 7B is a bar chart showing electrochemical signal generated by a hybridization probe having the nucleotide sequence

(SEQ ID NO. 6)

CTTCGTTCGATTGTC^{▾}TGGACTTATAATGCTGAACTTCTGGT

wherein the probe has a 15 mer nucleotide sequence that is non-complementary to a target sequence of a target nucleic acid.

FIG. 7C is a bar chart showing electrochemical signal generated by a hybridization probe having the nucleotide sequence

(SEQ ID NO. 7)

TCGTTCGATTGTC^{▾}TGGACTTATAATGCTGAACTTCTGGT

wherein the probe has a 13 mer nucleotide sequence that is non-complementary to a target sequence of a target nucleic acid.

FIG. 8A is an illustration showing the predicted folded structure of a hybridization probe for bird flu which has a predicted Tₘ of 44.0° C.

FIG. 8B is a bar chart showing post PCR electrochemical signal generated by the bird flu DNA hybridization probe having the nucleotide sequence illustrated in FIG. 8A.

FIG. 9A is an illustration showing the predicted folded structure of a second hybridization probe for bird flu which has a predicted Tₘ of 45.3° C.

FIG. 9B is a bar chart showing the post PCR electrochemical signal generated by the second bird flu DNA hybridization probe having the nucleotide sequence illustrated in FIG. 9A.

FIG. 10 is a schematic showing a hybridization probe wherein the folded structure is an intramolecular triplex.

FIGS. 11A and 11B are illustrations of the structures of base pairing that occurs when triplexes form with a protonated cytosine (C+) nucleobase (FIG. 11A) and when the pseudoisocytosine nucleobase, also referred to herein as a J or J-base is substituted for the protonated cytosine nucleobase (FIG. 11B).

FIG. 12 is a schematic of a sealed electrochemical chamber which can be used for elevated temperature measurements.

### DESCRIPTION OF VARIOUS EMBODIMENTS

For the purposes of interpreting of this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example to understand the document where the term is originally used). The use of “or” herein means “and/or” unless stated otherwise or where the use of “and/or” is clearly inappropriate. The use of “a” herein means “one or more” unless stated otherwise or where the use of “one or more” is clearly inappropriate. The use of “comprise,” “comprises,” “comprising” “include,” “includes,” and “including” are interchangeable and not intended to be limiting. Furthermore, where the description of one or more embodiments uses the term “comprising,” those skilled in the art would understand that in some specific instances, the embodiment or embodiments can be alternatively described using language “consisting essentially of” and/or “consisting of.”

As used herein, “capture probe” refers to a nucleobase polymer that is surface bound. The capture probe can be a nucleic acid (e.g. DNA or RNA), a nucleic acid analog (e.g. locked nucleic acid (LNA)), a nucleic acid mimic (e.g. peptide nucleic acid (PNA)) or a chimera.

As used herein, “chimera” refers to a nucleobase polymer comprising two or more linked subunits that are selected from different classes of subunits. For example, a PNA/DNA chimera would comprise at least one PNA subunit linked to at least one 2′-deoxyribonucleic acid subunit (For exemplary methods and compositions related to PNA/DNA chimera preparation See: WO96/40709). Exemplary component subunits of a chimera are selected from the group consisting of PNA subunits, naturally occurring amino acid subunits, DNA subunits, RNA subunits, LNA subunits and subunits of other analogues or mimics of nucleic acids.

As used herein, “flap” refers to a portion of a hybridization probe that is non-complementary to the target nucleic acid the probe is designed to determine.

As used herein, “hybridization probe” is a nucleobase polymer that can be cleaved by exonuclease activity of an enzyme at a site where the probe is hybridized to a complementary strand, said hybridization probe comprising a nucleobase sequence that is complementary to at least a portion of a target nucleic acid of interest in a sample. The hybridization probe can be a oligonucleotide, oligonucleotide analog or chimera so long as it is cleavable by exonuclease activity. In some embodiments, the nucleobase polymer can be a chimera that comprises all DNA subunits except for one LNA subunit. In some embodiments, the nucleobase polymer comprises a single LNA subunit that is situated one subunit removed (toward the 3′ end) from the 5′ end of that portion of the hybridization probe that is designed to hybridize to the target nucleic acid.

As used herein, “nucleobase polymer” refers to a polymer comprising a series of linked nucleobase containing subunits. Non-limiting examples of suitable polymers include oligodeoxynucleotides, oligoribonucleotides, peptide nucleic acids, nucleic acid analogs, nucleic acid mimics and chimeras.

As used herein, “peptide nucleic acid” or “PNA” refers to any polynucleobase strand or segment of a polynucleobase strand comprising two or more PNA subunits, including, but not limited to, any polynucleobase strand or segment of a polynucleobase strand referred to or claimed as a peptide nucleic acid in U.S. Pat. Nos. 5,539,082, 5,527,675, 5,623,049, 5,714,331, 5,718,262, 5,736,336, 5,773,571, 5,766,855, 5,786,461, 5,837,459, 5,891,625, 5,972,610, 5,986,053, 6,107,470 and 6,357,163. For the avoidance of any doubt, PNA is a nucleic acid mimic and not a nucleic acid or nucleic acid analog. PNA is not a nucleic acid since it is not formed from nucleotides. For the avoidance of doubt, PNA oligomers may include polymers that comprise one or more amino acid side chains linked to the backbone.

As used herein, “support”, “solid support” or “solid carrier” refers to any solid phase material. Solid support encompasses terms such as “resin”, “synthesis support”, “solid phase”, “surface” “membrane” and/or “support”. A solid support can be composed of organic polymers such as polystyrene, polyethylene, polypropylene, polyfluoro ethylene, polyethyleneoxy, and polyacrylamide, as well as co-polymers and grafts thereof. A solid support can also be inorganic, such as glass, silica, controlled-pore-glass (CPG), or reverse-phase silica. The configuration of a solid support can be in the form of beads, spheres, particles, granules, a gel, a membrane or a surface. Surfaces can be planar, substantially planar, or non-planar. Solid supports can be porous or non-porous, and can have swelling or non-swelling characteristics. A solid support can be configured in the form of a well, depression, tube, channel, cylinder or other container, vessel, feature or location.

As used herein, “target nucleic acid” refers to a nucleic acid molecule of interest. A sample can comprise more than one target nucleic acid molecule.

As says for the electrochemical detection of a PCR product are disclosed in Provisional U.S. Patent Application No. 60/877,610, filed on even date herewith (Attorney Docket No. 70043.0036USP1). This assay consists of a hybridization probe with, for example, a 15-mer 5′ flap that is non-complimentary to a target nucleic acid but is complimentary to an electrode confined capture probe. This 5′ flap comprises an electrochemical label. During the PCR process, a probe fragment comprising this 5′ flap can be cleaved by an enzyme having exonuclease activity, such as Taq Polymerase. The probe fragment can then hybridize to the electrode confined capture probe and generate signal. The intact (i.e. uncleaved) hybridization probe was found to not hybridize as efficiently to the capture probe as did the probe fragment. This phenomenon permits the monitoring of PCR without separation of the probe fragment from the intact hybridization probe in a one pot assay.

In the assays described herein, the intact or uncleaved hybridization probe can form a folded structure having a melting temperature (Tₘ) which is lower than the melting temperature of the duplex formed when the intact hybridization probe hybridizes to the target nucleic acid and higher than the melting temperature of the duplex formed when the probe fragment hybridizes to the capture probe. Without intending to be bound by any theory, it is believed that the folded structure of the intact hybridization probe at temperatures under which the probe fragment hybridizes to the capture probe substantially inhibits hybridization of the intact hybridization probe to the capture probe on the electrode surface thereby improving the signal to noise ratio of the assay.

1. PCR Assay Performed with a Hybridization Probe Comprising a 5′-Flap

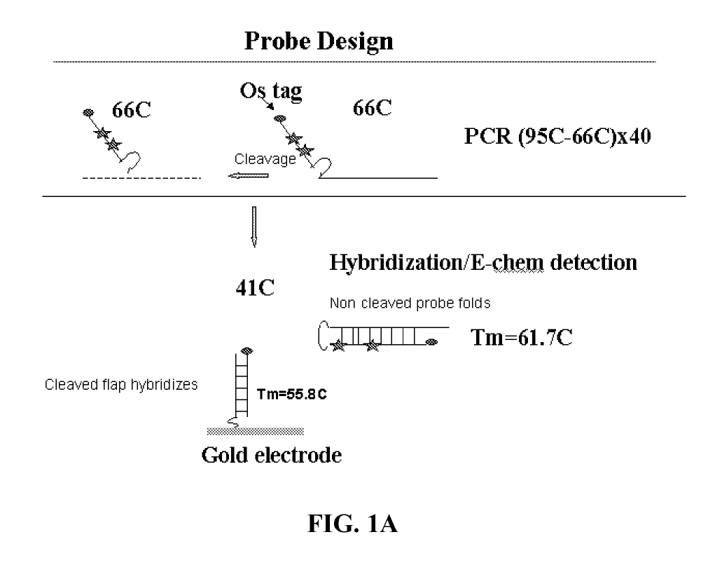

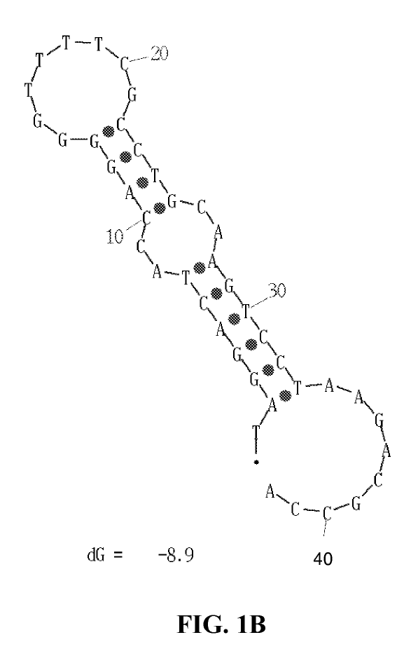

FIG. 1A is a schematic for the design of the components and the steps of an assay that uses a hybridization probe capable of forming a folded structure, wherein the hybridization probe can be hybridized to a target nucleic acid and wherein a portion of the hybridized probe is cleaved to form a labeled probe fragment that can be captured and detected on a surface (e.g. an electrode surface). With this assay format in mind, a set of hybridization probes with various predicted Tₘ values (e.g., 83.5° C., 61.7° C., 54.3° C. or 46° C.) for the folded structure were designed and evaluated. The hybridization probe with the highest Tₘ (=83.5° C.) corresponded to the perfect match between a 15 mer region of the 5′ flap and the remainder of the probe. The other probes contained mismatches which resulted in lower Tₘ values. The efficiency of the cleavage of these probes during PCR was evaluated using HPLC separation of the cleaved and intact hybridization probes as described in U.S. patent application Ser. No. 11/488,439, filed on Jul. 17, 2006. The HPLC column XTerroMSC18 (2.5 mm×50 mm) from Waters Corp. was equilibrated with 7% ACN+93% TEAA. A gradient elution (0.3 ml/min, 60 C) was performed in three steps: Step 1: 7% ACN+93% TEAA for 7 min. Step 2: 10% ACN+90% TEAA for 10 min. Step 3: 35% ACN+65% TEAA for 10 min. (ACN—Acetonitrile. TEAA—0.1M Triethanolamine—Acetic acid at pH 6.8). The hybridization probe with a Tₘ=61.7° C. for the folded structure showed 30% of cleavage efficiency and was selected for electrochemical detection of PCR.

FIG. 1B illustrates the predicted folded structure of the hybridization probe with a predicted Tₘ=61.7° C. This hybridization probe is suitable for determining the Listeria monocytogenesis hlyA gene in accordance with the assay illustrated in FIG. 1A. The probe illustrated in FIG. 1B has the following sequence:

(SEQ ID NO. 1)

TAGGACTACCAGGGGTTTTC^{▾}GCCTGCAAGTCCTAAGACGCCA

wherein the nucleobases illustrated in bold represents the 5′ flap and the ^{▾} symbol represents the site where cleavage by the exonucleoase activity is expected to be predominant.

Using this hybridization probe comprising a 5′ osmium electrochemical tag according to the assay illustrated in FIG. 1A, PCR of a fragment of Listeria monocytogenesis hlyA gene (i.e. the target nucleic acid) was performed. The PCR reaction was run for 10 min at 95° C., then (15 sec. at 95° C., 1 min at 63° C.)×40 cycles in PCR buffer A (Applied Biosystems, Catalog No. N808-0228) supplied with 6 mM MgCl₂. Primers and probe were at concentrations of 200 nM and 400 nM, respectively. This hybridization probe has a 19-mer 5′ flap which is partially complementary to the internal part of the probe (see FIG. 1B). During PCR at the annealing-extension temperature (i.e., 66° C.), the hybridization probe should be substantially unfolded since the assay temperature is above the predicted Tₘ of the folded structure. This permits the hybridization probe to hybridize to the target nucleic acid. Once hybridized, the enzyme having exonuclease activity cleaves the hybridized hybridization probe to thereby produce the probe fragments during the PCR reaction. After completion of PCR, the temperature of the sample is dropped to 41° C. to allow hybridization of the probe fragment(s) to the capture probe(s). Under these conditions, any intact (i.e. uncleaved) hybridization probe still present in the sample should form the predicted folded structure as shown in FIG. 1B such that the 5′ flap is not substantially accessible to the surface bound capture probes.

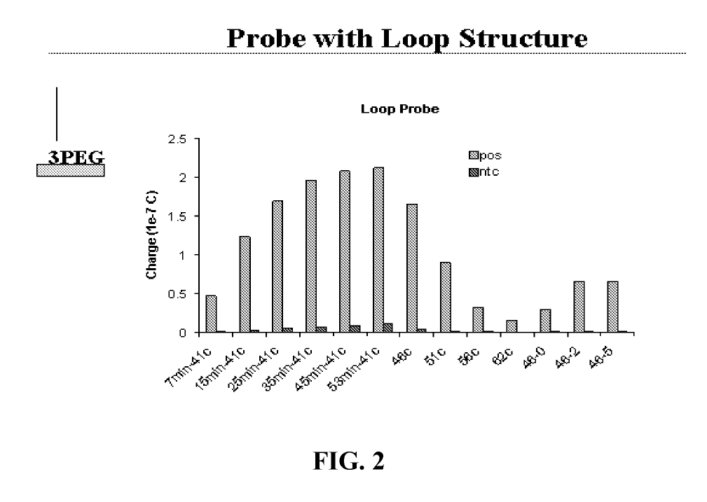

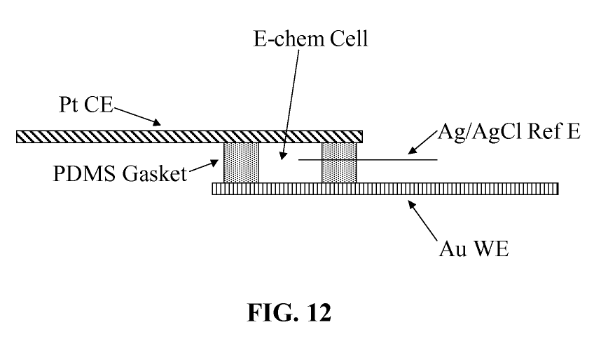

The results of the electrochemical measurements for this assay are shown in FIG. 2. After 40 cycles of PCR, both the positive (pos) and no template control (ntc) reaction mixes were placed into an electrochemical cell sandwiched between two heating plates. The electrochemical cell used in this experiment is depicted in FIG. 12. As shown in FIG. 12, this cell includes a working electrode (WE) and a counter electrode (CE) having diameters of 2 mm. The platinum counter-electrode (CE) was made by sputter coating a 2000 Angstrom thick platinum layer on a silicon wafer having a Cr adhesion layer. The gold counter-electrode (CE) was made by sputter coating a 2000 Angstrom thick gold layer on a silicon wafer having a Cr adhesion layer. The reference electrode was a 0.5 mm diameter Ag/AgCl wire. As can be seen from the results shown in FIG. 2, the intact hybridization probe hybridizes 20 to 30 times less effectively than the cleaved probe fragments.

2. PCR Assay Performed with a Hybridization Probe Comprising Interacting 5′ and 3′ Flaps

A hybridization probe with complementary 5′ and 3′ flaps that can be used for determining bird flu virus RNA has the sequence:

(SEQ ID NO: 2)

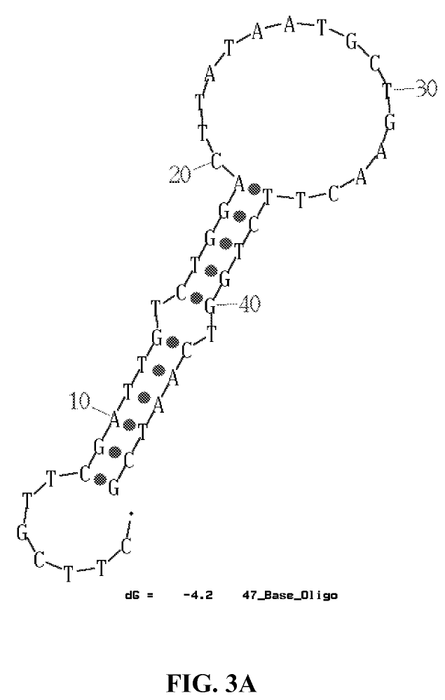

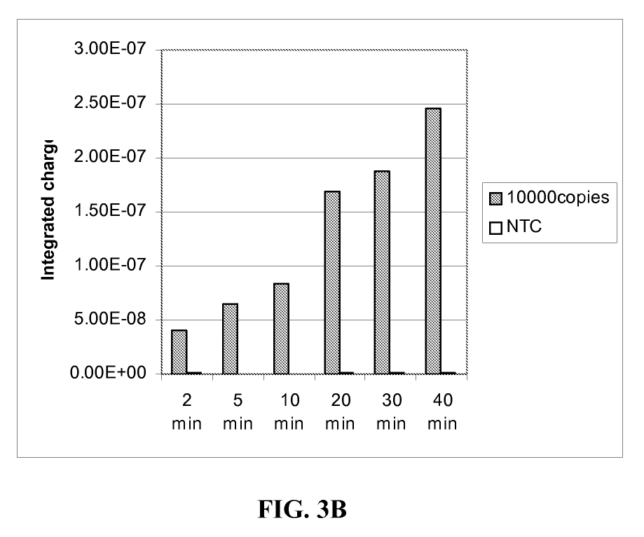

CTTCGTTCGATTGTC^{▾}TGGACTTATAATGCTGAACTTCTGGTCAATCG

wherein the nucleobases illustrated in bold represent the 5′ and 3′ flaps and the ^{▾} symbol represents the site where cleavage by the exonucleoase activity is expected to be predominant. The predicted Tₘ of the folded structure of this probe is 43.9° C. FIG. 3A illustrates the predicted folded structure of this hybridization probe.

PCR assays were performed using this hybridization probe. After 40 cycles of PCR (extension and annealing at 60° C.) in environmental master mix the temperature was shifted to 28° C. The environmental master mix included 100 mM KCl, 100 mM Tris pH 8, 8 mM MgCl₂, 100 μM dntps and 0.3 units/μL gold ampliTaq. At this temperature, the probe fragment generated by exonuclease activity on the hybridized hybridization probe, can anneal to the surface bound capture probe (Tₘ=32° C.). Under these conditions, any intact (i.e. uncleaved) hybridization probe still present in the sample should form the predicted folded structure as shown in FIG. 3A (Tₘ=43.9° C.) such that the 5′ flap is not substantially accessible to the surface bound capture probes.

FIG. 3B is a bar chart showing electrochemical signal generated on the surface electrode after PCR for the hybridization probe illustrated in FIG. 3A at various time points. PCR was performed both in the presence of 10000 copies of the target nucleic acid and in the absence of target nucleic acid (no target control or NTC). As shown in FIG. 3B, the electrochemical data indicate an approximately 100 fold discrimination between hybridization efficiencies of the two assays.

This assay was repeated with another probe having the nucleobase sequence:

(SEQ ID NO: 3)

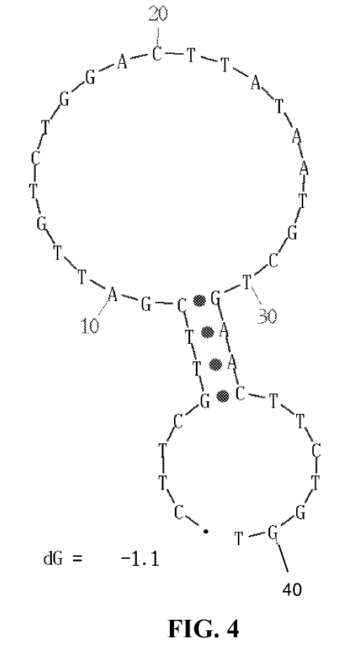

CTTCGTTCGATTGTC^{▾}TGGACTTATAATGCTGAACTTCTGGT

wherein the nucleobases illustrated in bold represent a 5′ flap and the ^{▾} symbol represents the site where cleavage by the exonucleoase activity is expected to be predominant. FIG. 4 illustrates the predicted folded structure of this hybridization probe. This folded structure has a predicted Tₘ of 34.2° C. All Tₘ and mFold analyses were done for 5 mM MgCl₂ media which corresponds to the ionic strength of environmental master mix.

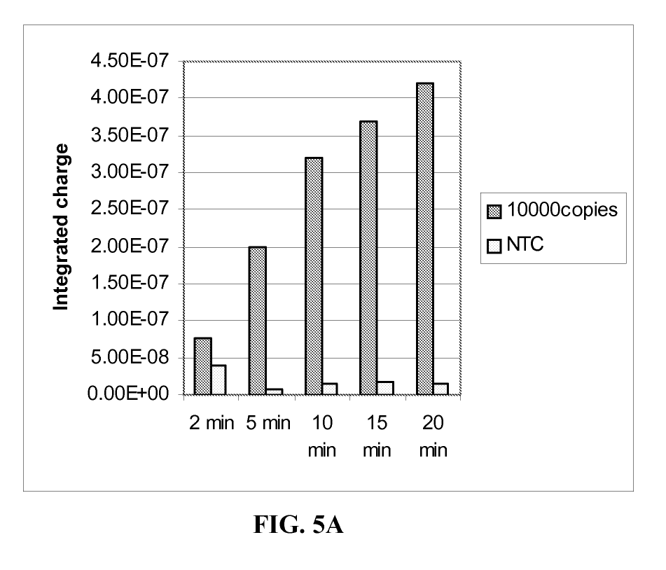

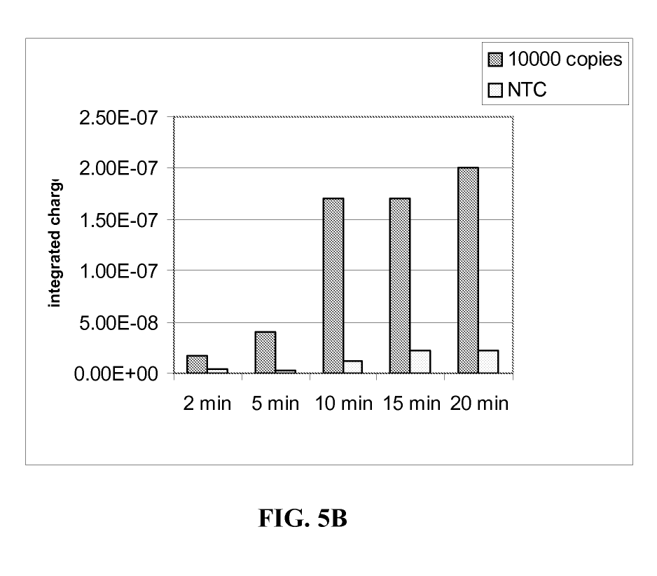

PCR assays were performed to compare the efficiency of electrochemical detection using the hybridization probe illustrated in FIG. 3A as compared with the hybridization probe illustrated in FIG. 4. FIG. 5A is a bar chart showing electrochemical signal for the PCR assay performed with the hybridization probe illustrated in FIG. 3A. FIG. 5B is a bar chart showing electrochemical signal for the PCR assay performed with the hybridization probe illustrated in FIG. 4. The results presented in FIG. 5A show approximately a 2 to 3 times better discrimination for the hybridization probe illustrated in FIG. 3A as compared with the hybridization probe illustrated in FIG. 4.

### 3. The Effect of 3′ Flap Length.

Additional experiments were conducted to elucidate the effect of the length of the 3′ flap on the stability of the folded structure. The hybridization probes used for these experiments included the probe illustrated in FIG. 3 which had a 3′ flap of 6 nucleobases in length and a similar probe having an elongated 3′ flap of 9 nucleobases in length. The structure of a hybridization probe with the longer 9 nucleobase 3′ flap has the sequence:

(SEQ ID NO: 4)

CTTCGTTCGATTGTC^{▾}TGGACTTATAATGCTGAACTTCTGGTCAATCG
AAC

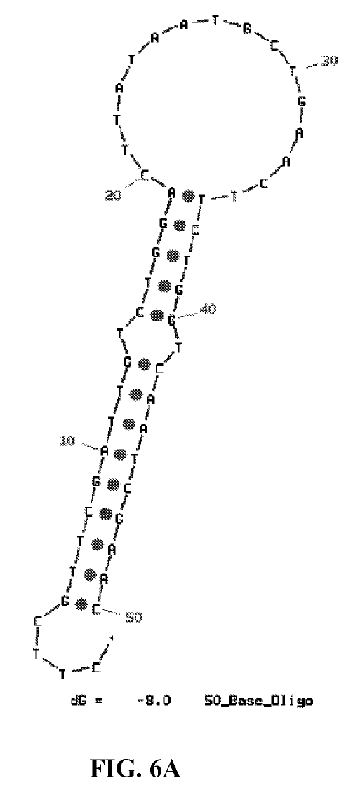

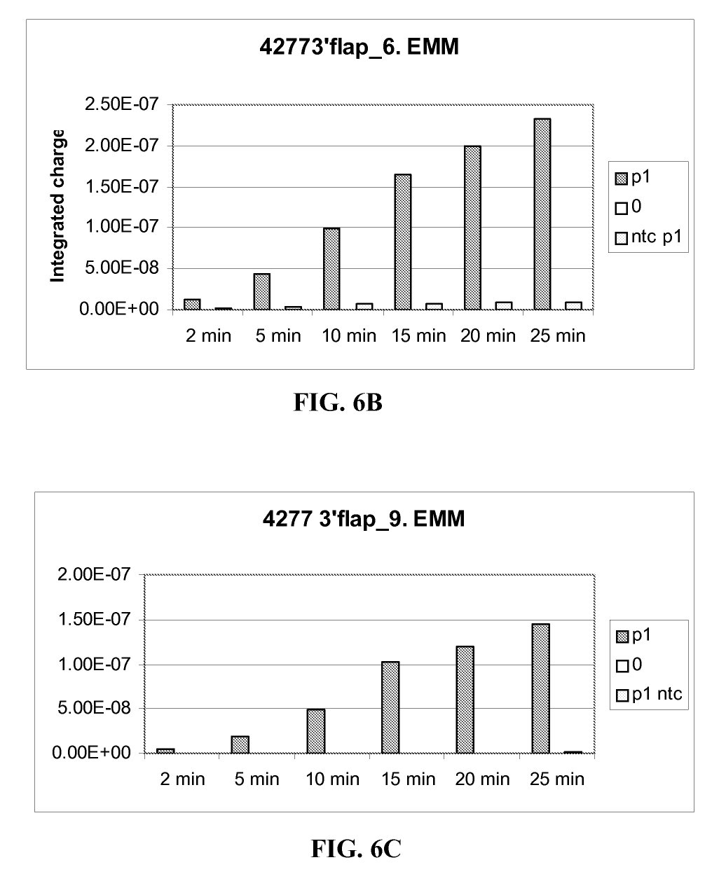

This hybridization probe has the predicted folded structure set forth in FIG. 6A and a predicted Tₘ of 53.1° C. The nucleobases illustrated above in bold represent the 5′ and 3′ flaps and the ^{▾} symbol represents the site where cleavage by the exonucleoase activity is expected to be predominant. In this hybridization probe, the underlined C nucleobase that is adjacent to the illustrated cleavage site is an LNA subunit. All other subunits of the hybridization probe are DNA.

The probe with the 9 nucleobase long 3′ flap has a predicted melting temperature of approximately 53.1° C. whereas the probe with the shorter 6 nucleobase long 3′ flap has a predicted melting temperature of approximately 43.9° C. The results of electrochemical detection after performing a PCR assay for the hybridization probes illustrated in FIG. 3A and FIG. 6A are shown in FIGS. 6B and 6C, respectively. The electrochemical analysis of the post PCR hybridization reactions was carried out at 32° C. on a gold surface. Due to the increased length of the 3′ flap, the probe with the 9 nucleobase long 3′ flap has a predicted more stable structure (i.e., a higher folding Tₘ) which apparently resulted in better discriminating ability.

### 4. The Effect of 5′ Flap Length

Additional experiments were conducted to determine the effect of the length of the 5′ flap on assay performance. For these experiments, hybridization probes having 19 mer, 15 mer and 13 mer 5′ flaps directed to bird flu virus were evaluated. These probes did not include a 3′ flap. The probes had the following nucleotide sequences:

19-mer 5′ flap

(SEQ ID NO: 5)

GTTACTTCGTTCGATTGTC^{▾}TGGACTTATAATGCTGAACTTCTGGT
15-mer 5′ flap

(SEQ ID NO: 6)

CTTCGTTCGATTGTC^{▾}TGGACTTATAATGCTGAACTTCTGGT
13-mer 5′ flap

(SEQ ID NO: 7)

TCGTTCGATTGTC^{▾}TGGACTTATAATGCTGAACTTCTGGT

The nucleobases illustrated above in bold in these sequences represent the 5′ flap and the ^{▾} symbol represents the site where cleavage by the exonucleoase activity is expected to be predominant. In these hybridization probes, the underlined C nucleobase that is adjacent to the illustrated cleavage site is an LNA subunit. All other subunits of these hybridization probes are DNA.

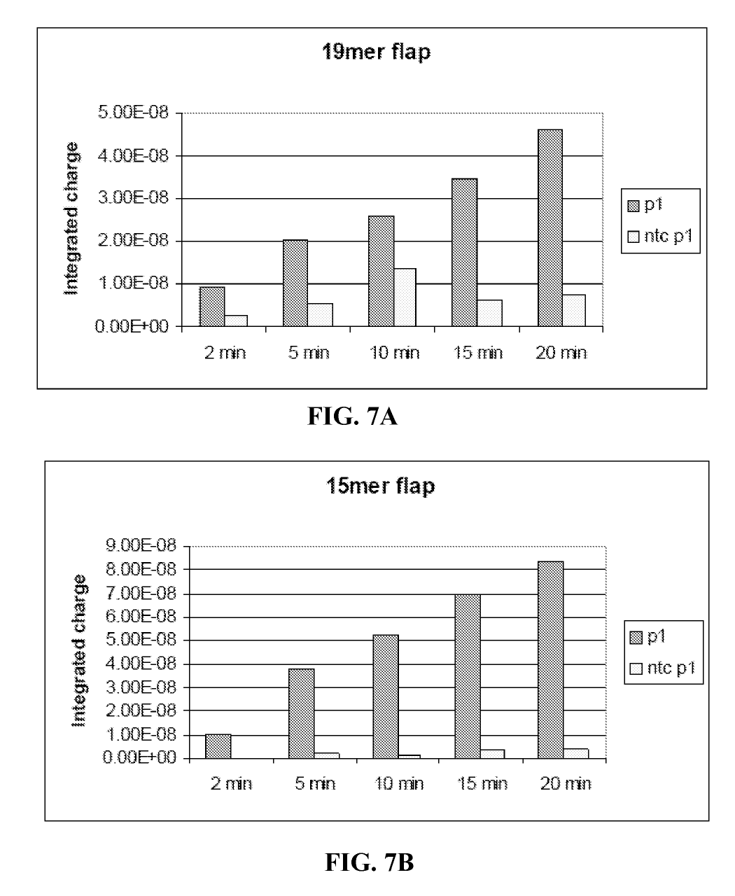

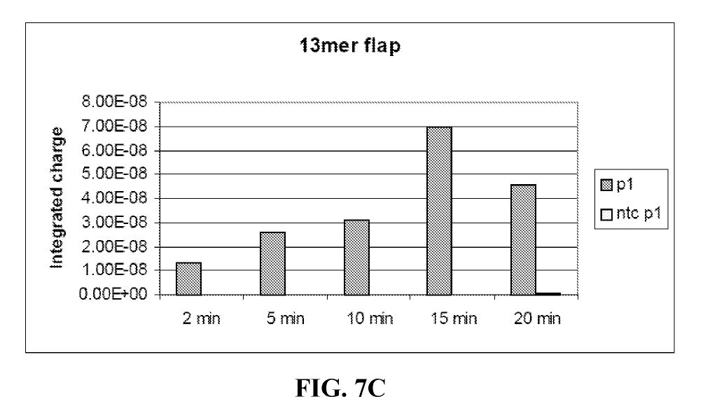

The results of electrochemical detection after performing the PCR using each of the hybridization probes having the 19 mer, 15 mer and 13 mer 5′ flaps are shown in FIGS. 7A, 7B and 7C, respectively. The post PCR mix was hybridized on gold electrodes at 41° C., 35° C. and 31° C. for the 19 mer, 15 mer and 13 mer 5′ flaps, respectively (i.e., 8° C. lower than the predicted Tₘ of each of the predicted folded structures). As can be seen from these results, the hybridization probe having the 13 mer 5′ flap exhibited better assay performance than did the hybridization probes having the longer 15 mer and 19 mer 5′ flaps.

### 5. Additional Bird Flu PCR Assays

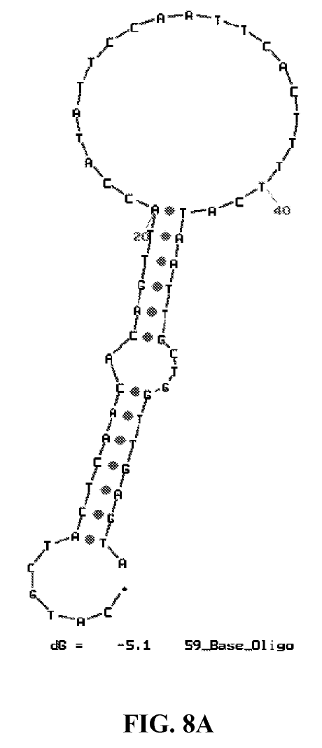

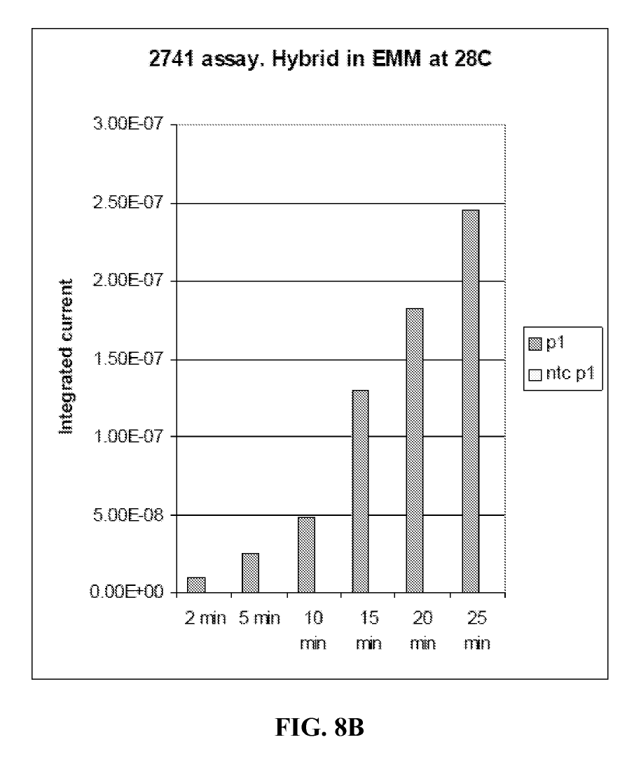

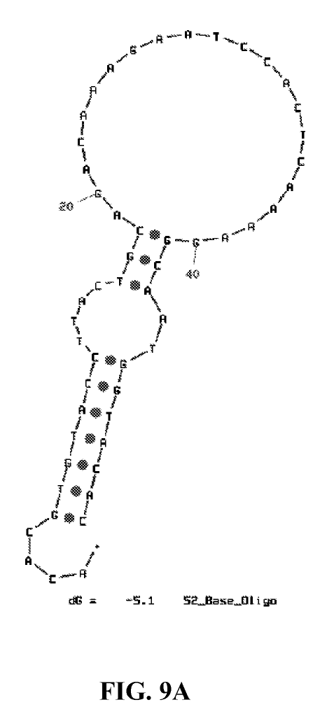

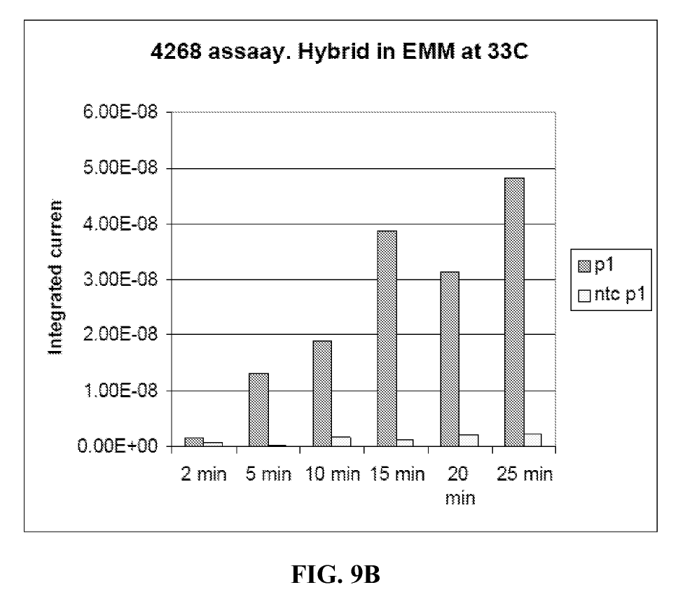

Two additional bird flu PCR assays, which were directed to different regions of the hemaglutinin gene of the bird flu virus, were conducted. Both hybridization probes were designed with a 3′ flap which resulted in a predicted Tₘ for the folded structure of approximately 44-45° C. The predicted folded structures for these two probes are illustrated in FIG. 8A and FIG. 9A. Templates that served as the target nucleic acid for these assays were synthetic DNAs of about 100 bases in length. Post PCR hybridization/detection was conducted on gold electrodes using environmental master mix at temperatures 10 to 14° C. below the predicted Tₘ of the probe fragment/capture probe hybrid.

The nucleobase sequence of the first hybridization probe used in these experiments is:

(SEQ ID NO: 8)

CATGCTACTCAACAC^{▾}AGTTACCATATTCCAATTCACTTTTCATAATTGC
TGGTTGAGTA

This hybridization probe had a predicted melting point (Tₘ) of 44.0° C. for the folded structure. The predicted folded structure of this probe is set forth in FIG. 8A. The capture probe used with this probe was a 15 mer oligomer having a structure as set forth below:

| | |
|---|---|
| GTGTTGAGTAGCATG | (SEQ ID NO: 9) |

The nucleobase sequence of the second hybridization probe used in these experiments is:

(SEQ ID NO: 10)

ACACGTGTACCTTAC^{▾}TGCAGACAAAGAATCCACTCAAAAGGCAATGG
TACAC

This hybridization probe had a predicted melting point (Tₘ fold) of 45.2° C. for the folded structure. The predicted folded structure of this probe is set forth in FIG. 9A. The capture probe used with this hybridization probe had a structure as set forth below:

| | |
|---|---|
| GTAAGGTACACGTGT | (SEQ ID NO: 11) |

For these two hybridization probes, the nucleobases illustrated above in bold represent the 5′ and 3′ flaps and the ^{▾} symbol represents the site where cleavage by the exonucleoase activity is expected to be predominant. In these hybridization probes, the underlined A (first probe) and underlined T (second probe) nucleobase that is adjacent to the illustrated cleavage site is an LNA subunit. All other subunits of these hybridization probes are DNA.

These hybridization and capture probes were used in PCR assays as discussed herein. Post PCR electrochemical detection of bird flu DNA using the first hybridization probe is shown in FIG. 8B. Post PCR electrochemical detection of bird flu DNA using second hybridization probe is shown in FIG. 9B.

Although hybridization probes which adopt stem loop type conformations are disclosed above, hybridization probes adopting other conformations upon folding can also be employed. Such structures include hairpin, internal loop, bulge, branched, cloverleaf and pseudoknot structures. Examples of other folded structures that can be used in the practice of the methods and kits disclosed herein can be found in U.S. Pat. No. 7,118,860 B2.

### 6. Hybridization Probes Comprising Triplex Structures

In some embodiments, the hybridization probe can adopt an intramolecular triplex conformation. An example of a hybridization probe which can form an intramolecular triplex structure is set forth below:

(SEQ ID NO: 12)

TTJJTAGATCCTT-[Probe Sequence]-AAGGA

In the above sequence, “J” represents a pseudoisocytosine nucleobase, “Probe Sequence” represents the portion of the probe which is designed to hybridize sequence specifically to the target nucleic acid.

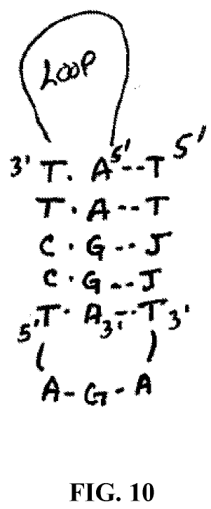

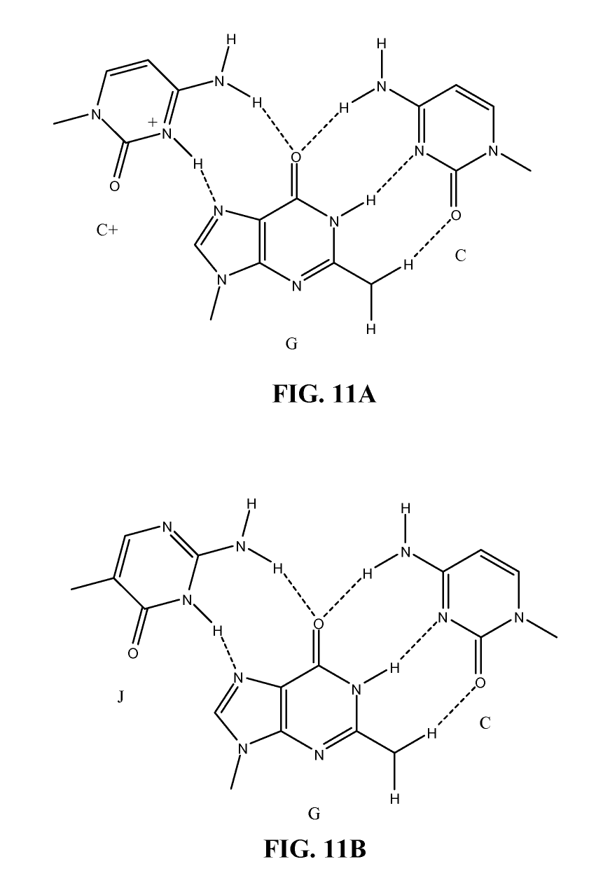

It is anticipated that a hybridization probe of this general configuration can adopt an intramolecular triplex conformation as its folded structure as illustrated in FIG. 10 wherein “- -” represents Hoogsteen hydrogen bonds, “•” represents Watson-Crick base pairs and “loop” comprises the portion of the probe which hybridizes to the target nucleic acid (i.e., the “probe sequence”). Triplex structures of this type are disclosed in Petrov et al., “The Triplex-Hairpin Transition in Cytosine-Rich DNA”, Biophysical Journal, Vol. 87, 3954-3973 (December 2004). These structures involve the formation of T•A--T and C•G--C⁺ triplexes wherein C⁺ represents a cytosine residue protonated at the N3 position. With reference to FIG. 11 and Egholm et al., “Efficient pH-independent sequence-specific DNA bindng by pseudoisocytosine-containing bis-PNA”, Nucl. Acids Res., Vol. 23, No. 2. 217-222 (1995), pseudoisocytosine can be substituted where a protonated cytosine nucleobase (C+) is need to produce a triplex. In this way, the triplex structure can be formed in a pH independent manner.

In the sequence set forth above and structure illustrated in FIG. 10, pseudoisocytosine is substituted for those cytosine nucleobases that would be protonated. Consequently this substitution of J-base for C+ at the positions indicated permits the triplex to form at physiological pH. Hybridization probes that form intramolecular triplexes are expected to form stable folded structures (i.e. triplex structures) that would substantially inhibit their interaction with the surface bound capture probes.

### 7. Labels, Probes & Primers

Any known electrochemical moiety can be used as a label on the cleaved portion of the hybridization probe. Exemplary electrochemical labels which may be used include bis(2,2′-bipyridyl)imidizolylchloroosmium (II) [salt]. This label gives a good Eₒ of 0.165 vs Ag/AgCl and has good solubility properties for synthesis and purification. Other exemplary labels include ferrocene as well as the labels disclosed in U.S. patent application Ser. No. 11/488,439 filed on Jul. 17, 2006. Moreover, the electrochemical label can be any moiety that can transfer electrons to or from an electrode. Exemplary electrochemical labels include transition metal complexes. Suitable transition metal complexes include, for example, ruthenium²⁺(2,2′-bipyridine)₃ (Ru(bpy)₃²⁺), ruthenium²⁺(4,4′-dimethyl-2,2′-bipyridine)₃ (Ru(Me²-bpy)₃²⁺), ruthenium²⁺(5,6-dimethyl-1,10-phenanthroline)₃ (Ru(Me₂-phen)₃²⁺), iron²⁺(2,2′-bipyridine)₃ (Fe(bpy)₃²⁺), iron²⁺(5-chlorophenanthroline)₃ (Fe(5-Cl-phen)₃²⁺), osmium²⁺(5-chlorophenanthroline)₃ (Os(5-Cl-phen)₃²⁺), osmium²⁺(2,2′-bipyridine)₂ (imidazolyl), dioxorhenium¹⁺ phosphine, and dioxorhenium¹⁺ pyridine (ReO₂(py)₄¹⁺). Some anionic complexes useful as mediators are: Ru(bpy)((SO₃)₂-bpy)₂²⁻ and Ru(bpy)((CO₂)₂-bpy)₂²⁻ and some zwitterionic complexes useful as mediators are Ru(bpy)₂ ((SO₃)₂-bpy) and Ru(bpy)₂((CO₂)₂-bpy) where (SO₃)₂-bpy₂- is 4,4′-disulfonato-2,2′-bipyridine and (CO₂)₂-bpy₂- is 4,4′-dicarboxy-2,2′-bipyridine. Suitable substituted derivatives of the pyridine, bypyridine and phenanthroline groups may also be employed in complexes with any of the foregoing metals. Suitable substituted derivatives include but are not limited to 4-aminopyridine, 4-dimethylpyridine, 4-acetylpyridine, 4-nitropyridine, 4,4′-diamino-2,2′-bipyridine, 5,5′-diamino-2,2′-bipyridine, 6,6′-diamino-2,2′-bipyridine, 4,4′-diethylenediamine-2,2′-bipyridine, 5,5′-diethylenediamine-2,2′-bipyridine, 6,6′-diethylenediamine-2,2′-bipyridine, 4,4′-dihydroxyl-2,2′-bipyridine, 5,5′-dihydroxyl-2,2′-bipyridine, 6,6′-dihydroxyl-2,2′-bipyridine, 4,4′,4″-triamino-2,2′,2″-terpyridine, 4,4′,4″-triethylenediamine-2,2′,2″-terpyridine, 4,4′,4″-trihydroxy-2,2′,2′-terpyridine, 4,4′,4″-trinitro-2,2′,2″-terpyridine, 4,4′,4″-triphenyl-2,2′,2″-terpyridine, 4,7-diamino-1,10-phenanthroline, 3,8-diamino-1,10-phenanthroline, 4,7-diethylenediamine-1,10-phenanthroline, 3,8-diethylenediamine-1,10-phen anthroline, 4,7-dihydroxyl-1,10-phenanthroline, 3,8-dihydroxyl-1,10-phenanthroline, 4,7-dinitro-1,10-phenanthroline, 3,8-dinitro-1,10-phenanthroline, 4,7-diphenyl-1,10-phenanthroline, 3,8-diphenyl-1,10-phenanthroline, 4,7-disperamine-1,10-phenanthroline, 3,8-disperamine-1,10-phenanthroline, dipyrido[3,2-a:2′,2′-c]phenazine, and 6,6′-dichloro-2,2′-bipyridine, among others.

Although electrochemical detection is exemplified above, the disclosed methods are also applicable to the detection of nucleic acids by other detection techniques, such as fluorescence detection. Moreover, the detectable label on the hybridization probe can be any moiety which is capable of being detected and/or quantitated. Exemplary labels include electrochemical, luminescent (e.g., fluorescent, luminescent, or chemiluminescent) and calorimetric labels.

The primers and probes used herein may have any of a variety of lengths and configurations. For example, the primers may be from 18 to about 30 subunits in length or from 20 to 25 subunits in length. Primers need not be limited to DNA or RNA oligonucleotides but they must be extendable by a polymerase. Longer or shorter length primers can also be used.

The length of the region of the hybridization probe which binds to the target nucleic acid can be from 8 to 30 subunits in length whereas the length of the region of the hybridization probe which does not bind to the target nucleic acid (i.e., the 5′ flap) can have a length of 2 to 40 subunits or from 8 to 30 subunits. Hybridization probes having longer or shorter regions than those exemplified above can also be used.

The PCR primers may be designed to bind to and produce an amplified product of any desired length, usually at least 30 or at least 50 nucleotides in length and up to 200, 300, 500, 1000, or more nucleotides in length. The probes and primers may be provided at any suitable concentrations. For example, forward and reverse primers may be provided at concentrations typically less than or equal to 500 nM, such as from 20 nM to 500 nm, or 50 to 500 nM, or from 100 to 500 nM, or from 50 to 200 nM. Probes are typically provided at concentrations of less than or equal to 1000 nM, such as from 20 nM to 500 nm, or 50 to 500 nM, or from 100 to 500 nM, or from 50 to 200 nM. Exemplary conditions for concentrations of NTPs, enzyme, primers and probes can also be found in U.S. Pat. No. 5,538,848 which is incorporated herein by reference in its entirety, or can be achieved using commercially available reaction components (e.g., as can be obtained from Applied Biosystems, Foster City, Calif.).

A plurality of complementary capture probes, each having a characteristic sequence, may also be used in an array format. For example, an array of capture oligonucleotides that hybridize to different hybridization probe fragments may be used to localize and capture individual tag sequences in a plurality of discrete detection zones.

The methods described herein can be used to detect target nucleic acid in real time. For example, the solid support can be in contact with the solution in which nucleic acid amplification is occurring and the process monitored during PCR (i.e. real-time detection). Alternatively, the solid support can be in contact with the solution after the PCR process is complete (i.e., endpoint detection). In some embodiments, the PCR assay can be monitored during PCR (real-time) and after the process in completed (end-point). PCR assays can be performed using traditional PCR formats as well as Fast PCR formats, asymmetric PCR formats and asynchronous PCR formats.

The method described herein allows for a homogenous PCR assays where detection of the surface hybridization of the probe fragment of the hybridization probe indicates the presence of a target nucleic acid in a sample.

While the foregoing specification teaches the principles of the present invention, with examples provided for the purpose of illustration, it will be appreciated by one skilled in the art from reading this disclosure that various changes in form and detail can be made without departing from the true scope of the invention.

What is claimed is:

1. A method for detecting a target nucleic in a sample, the method comprising:
melting the sample by heating the sample to a first temperature, wherein the sample comprises:
a primer which hybridizes to at least a portion of the target nucleic acid;
a hybridization probe comprising first and second regions, wherein the first region hybridizes to at least a portion of the target nucleic acid and the second region does not hybridize to the target nucleic acid and wherein the second region comprises a detectable label; and
a polymerase and an enzyme comprising an exonuclease activity, wherein the polymerase extends the hybridized primer in the direction of the hybridized probe and the exonuclease activity of the enzyme cleaves the hybridized probe to thereby release a probe fragment comprising the second region of the probe and the detectable label; and
wherein the first temperature is above the Tₘ of the primer and double stranded nucleic acid present in the sample;
subsequently annealing the sample by reducing the temperature to a second temperature lower than the first temperature to allow the primer and the hybridization probe to each hybridize to a single stranded portion of the target nucleic acid in the sample; and
subsequently elongating the primer by allowing the polymerase to extend the primer hybridized to the target nucleic acid at a third temperature;
allowing the exonuclease activity of the enzyme to cleave the hybridization probe thereby releasing the probe fragment;
optionally repeating melting, annealing and elongating at least once;
contacting the sample with a surface of a solid support, wherein the surface of the solid support comprises one or more capture probes which hybridize to at least a portion of the second region of the probe fragment;
allowing the capture probes to hybridize to at least a portion of the probe fragment present in the sample at a fourth temperature, wherein the fourth temperature is lower than the second and third temperatures; and
detecting label on the surface of the solid support;
wherein at least one portion of the hybridization probe hybridizes to another portion of the hybridization probe to thereby form a folded structure and wherein the melting temperature (Tₘ) of the folded structure is lower than the third temperature and higher than the fourth temperature.

2. The method of claim 1, wherein the polymerase and the enzyme comprising an exonuclease activity are the same molecule.

3. The method of claim 1, wherein the melting temperature (Tₘ) of the folded structure is between 41° C. and 66° C.

4. The method of claim 1, wherein the melting temperature (Tₘ) of the folded structure is between 58° C. and 63° C.

5. The method of claim 1, wherein the second region of the probe fragment to which the capture probe hybridizes is substantially not accessible to the capture probe in the corresponding folded structure.

6. The method of claim 1, wherein the third temperature is greater than or equal to the second temperature and less than the first temperature.

7. The method of claim 1, wherein the second temperature and the third temperature are the same.

8. The method of claim 1, wherein the hybridization probe further comprises a third region adjacent the first region and opposite the second region, wherein the third region does not hybridize to the target nucleic acid.

9. The method of claim 8, wherein at least a portion of both the second region and the third region of the hybridization probe are hybridized to another portion of the hybridization probe to form the folded structure.

10. The method of claim 8, wherein at least a portion of third region hybridizes to at least a portion of the second region to form the folded structure.

11. The method of claim 5, wherein at least a portion of the second region of the hybridization probe is hybridized to another portion of the hybridization probe to form the folded structure.

12. The method of claim 1, wherein the polymerase is a thermostable enzyme.

13. The method of claim 12, wherein the thermostable enzyme is Taq polymerase.

14. The method of claim 1, wherein the surface of the solid support comprises an electrode and wherein the detectable label is a moiety that can transfer electrons to or from the electrode.

15. The method of claim 14, wherein the detectable label is a Ferrocene moiety.

16. The method of claim 14, wherein the surface of the solid support comprises gold.

17. The method of claim 1, wherein the solid support comprises a plurality of interdigitated plates forming a flow channel, wherein at least some of the surfaces of the plates comprise capture probes, and wherein contacting the sample with a surface of a solid support comprises flowing the sample through the flow channel.

18. The method of claim 17, wherein the surfaces of the plates comprise electrodes.

19. The method of claim 18, wherein the surfaces of alternating plates comprise capture probes.

20. The method of claim 1, wherein the folded structure comprises an intramolecular triplex structure.

21. The method of claim 1, wherein melting, annealing and elongating are performed multiple times in a series of cycles.

22. The method of claim 21, wherein detecting label on the surface of the solid support occurs after the last melting, annealing and elongating cycle.

23. The method of claim 1, wherein the sample is in contact with the surface of the solid support during melting, annealing and elongating and wherein detecting occurs multiple times during the method and/or after the last melting, annealing and elongating cycle.

24. A kit for detecting a target nucleic acid in a sample comprising:
a hybridization probe comprising a first region which hybridizes to at least a portion of the target nucleic acid and a second region comprising a detectable label, wherein the second region does not hybridize to the target nucleic acid and wherein an exonuclease enzyme can cleave the hybridization probe when hybridized to the target nucleic acid to thereby produce a probe fragment comprising the second region and the detectable label;
a solid support comprising a capture probe on a surface thereof, wherein the capture probe hybridizes to the second region of the probe fragment;
optionally, a primer which hybridizes to at least a portion of the target nucleic acid; and
optionally, a polymerase and an enzyme comprising an exonuclease activity wherein the polymerase extends the hybridized primer in the direction of the hybridized probe and the exonucleoase activity of the enzyme cleaves the hybridized probe to thereby release a probe fragment comprising the second region of the probe and the detectable label;
wherein at least one portion of the hybridization probe hybridizes to another portion of the hybridization probe to thereby form a folded structure and wherein the melting temperature (Tₘ) of the folded structure is lower than the melting temperature of the duplex formed when the hybridization probe hybridizes to the target nucleic acid and higher than the melting temperature of the duplex that formed when the probe fragment hybridizes to the capture probe.

25. The kit of claim 24, wherein the surface of the solid support comprises an electrode and wherein the detectable label is a moiety that can transfer electrons to or from the electrode.

26. The kit of claim 25, wherein the detectable label is an electroactive Ferrocene moiety.

27. The kit of claim 25, wherein the surface of the solid support comprises gold.

28. The kit of claim 24, wherein the kit comprises a polymerase and an enzyme comprising an exonuclease activity.

29. The kit of claim 25, wherein the polymerase and the enzyme comprising an exonuclease activity are the same molecule.

30. The method of claim 2, wherein the polymerase is a thermostable enzyme.
